(19) United States
(12) Patent Application Publication
(10) Pub. No.: US 2025/0331983 A1
(43) Pub. Date: Oct. 30, 2025

(54) DYSPHONIA TREATMENT TOOL

(71) Applicant: IRYOHOJIN KENMUKAI, Kyotanabe-shi Kyoto (JP)

(72) Inventors: Yutaka TOYA, Kyotanabe-shi Kyoto (JP); Shinya HIROSHIBA, Kyotanabe-shi Kyoto (JP); Masahiro TANABE, Kyotanabe-shi Kyoto (JP)

(21) Appl. No.: 18/866,875

(22) PCT Filed: Apr. 11, 2023
(86) PCT No.: PCT/JP2023/014730

§ 371 Date: Nov. 18, 2024

(30) Foreign Application Priority Data
May 16, 2022 (JP) 2022-080262

(51) Int. Cl. A61F 2/20

(52) U.S. Cl. CPC A61F 2/20; A61F 2220/0008

(57) ABSTRACT

A dysphonia treatment tool includes a pair of clamping portions that have a front surface piece arranged on a front surface of an incised thyroid cartilage, a rear surface piece arranged on a rear surface of the incised thyroid cartilage, and an end surface piece connecting the front surface piece and the rear surface piece, engaged with each of the incision ends of the incised thyroid cartilage;, and a bridging portion that bridges the pair of clamping portions. Wherein on an opposite surface to the abutting surface of the end surface piece, the bridging portion is bridging in a region from the one end side to the other end side, or in a region from the one end side to a side in a midway from the one end side to the other end side on the opposite surface to abutting surface of the end surface piece.

### CROSS-REFERENCE TO RELATED APPLICATIONS AND PRIORITY

This patent application claims priority from International Patent Application No. PCT/JP2023/014730 filed Apr. 11, 2023 which claims priority from Japanese Patent Application No. 2022-080262 filed May 16, 2022. Each of these patent applications are herein incorporated by reference in its/their entirety.

### TECHNICAL FIELD

The present disclosure relates to a dysphonia treatment tool.

### BACKGROUND

A dysphonia treatment tool has been proposed for improving spasmodic dysphonia, in which the glottis is too closed and the vocal cords do not vibrate (Patent Document 1).

As shown in Patent Document 1, the dysphonia treatment tool includes a pair of clamping portions and a bridging portion bridging the pair of clamping portions. The pair of clamping portions have a front surface piece arranged on a front surface of the incised thyroid cartilage, a rear surface piece arranged on a rear surface of the incised thyroid cartilage, and an end surface piece connecting the front surface piece and the rear surface piece and abutting on an incision end surface of the incised thyroid cartilage, and are engaged with each of the mutually opposed incision ends of the incised thyroid cartilage.

### PRIOR ART REFERENCE

### Patent Document

Patent Document 1: Japanese Patent Publication No. 6564862

### SUMMARY

### Problems to be Solved

The dysphonia treatment tool of Patent Document 1 has a bent region that is deformed around an imaginary line extending in the width direction of the front surface piece, and a hole is formed in the bent region through which an integrated thread is passed. In the dysphonia treatment tool of Patent Document 1, it is disclosed that the stress applied to the bent region and be reduced and the breakage of the clamping portions can be suppressed by making the hole formed in the bent region a predetermined hole.

However, there is a demand for a dysphonia treatment tool such as that of Patent Document 1 to further suppress breakage of the clamping portions.

It is therefore an object of the present disclosure to provide a dysphonia treatment tool which is capable of suppressing breakage and has excellent durability.

### Means for solving the Problem

As a result of intensive research into achieving the above-mentioned object, the inventors have surprisingly found that the above-mentioned object can be achieved in a dysphonia treatment tool which includes a pair of clamping portions that have a front surface piece arranged on a front surface of an incised thyroid cartilage, a rear surface piece arranged on a rear surface of the incised thyroid cartilage, and an end surface piece connecting the front surface piece and the rear surface piece and abutting on an incision end surface of the incised thyroid cartilage, and are engaged with each of the mutually opposed incision ends of the incised thyroid cartilage; and a bridging portion that bridges the pair of clamping portions, by giving the bridging portion a specified structure, and have thus completed the present disclosure.

That is, The dysphonia treatment tool according to the present disclosure includes a pair of clamping portions that have a front surface piece arranged on a front surface of an incised thyroid cartilage, a rear surface piece arranged on a rear surface of the incised thyroid cartilage, and an end surface piece connecting the front surface piece and the rear surface piece and abutting on an incision end surface of the incised thyroid cartilage, and are engaged with each of the mutually opposed incision ends of the incised thyroid cartilage; and a bridging portion that bridges the pair of clamping portions. Wherein on an opposite surface to the abutting surface of the end surface piece, when a side where the end surface piece is joined to the front surface piece is defined as one end side, and a side where the end surface piece is jointed to the rear surface piece is defined as other end side, the bridging portion is bridging in a region from the one end side to the other end side, or in a region from the one end side to a side in a midway from the one end side to the other end side on the opposite surface to abutting surface of the end surface piece.

### Effects

In the present disclosure, a dysphonia treatment tool capable of suppressing breakage and having excellent durability is provided.

### BRIEF DESCRIPTION OF DRAWINGS

FIG. 1 shows a perspective view (FIG. 1(A)) of a dysphonia treatment tool according to the present embodiment, and a front view (FIG. 1(B)) of an abutting surface of an end surface piece that constitutes clamping portions of the dysphonia treatment tool and abuts on an incision end;

FIG. 2 shows an explanatory diagram that explains a mode of using the dysphonia treatment tool according to the present embodiment;

FIG. 3 shows an explanatory diagram that explains a method of using the dysphonia treatment tool according to the present embodiment;

FIG. 4 shows an explanatory diagram that explains a conventional dysphonia treatment tool and a method for using the same;

FIG. 5 shows a perspective view (FIG. 5(A)) of another dysphonia treatment tool according to the present embodiment, and a front view (FIG. 5(B)) of an abutting surface of an end surface piece that constitutes clamping portions of the dysphonia treatment tool and abuts on an incision end; and

FIG. 6 shows a perspective view (FIG. 6(A)) of another dysphonia treatment tool according to the present embodiment, and a front view (FIG. 6(B)) of an abutting surface of an end surface piece that constitutes clamping portions of the dysphonia treatment tool and abuts on an incision end;

### DETAILED DESCRIPTION

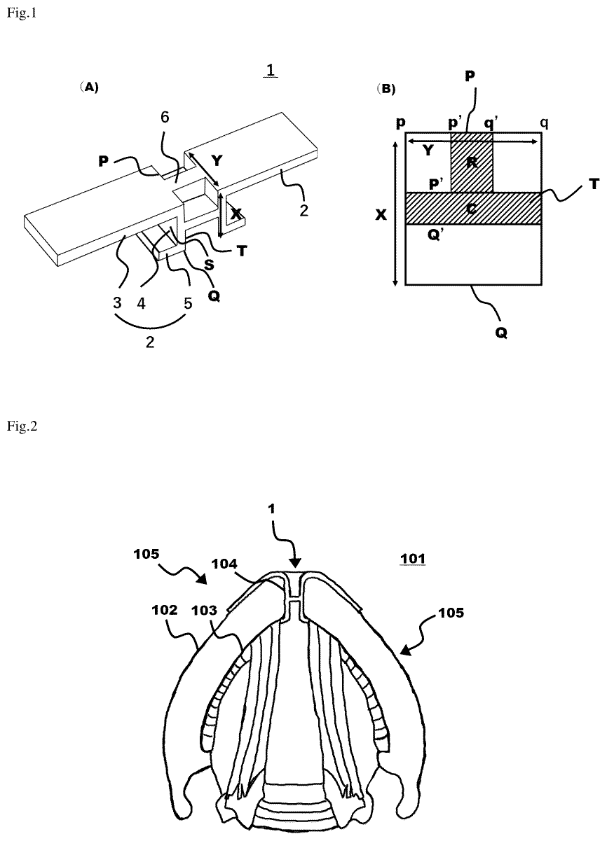

The dysphonia treatment tool according to the present embodiment will be described below with reference to FIG. 1 and FIG. 2. FIG. 1 shows a perspective view (FIG. 1(A) of the dysphonia treatment tool according to the present embodiment, and a front view (FIG. 1(B)) of an abutting surface S of an end surface piece 5 that constitutes clamping portions of the dysphonia treatment tool, which will be described later, and abuts on an incision end 105. FIG. 2 shows an explanatory diagram of the dysphonia treatment tool according to the present embodiment when used.

### Dysphonia Treatment Tool 1

The dysphonia treatment tool 1 shown in FIG. 1 and FIG. 2 includes a pair of clamping portions 2 and a bridging portion 6.

### Clamping Portion 2

As shown in FIG. 1 and FIG. 2, the pair of clamping portions 2 has a front surface piece 3 arranged on a front surface 102 of an incised thyroid cartilage 101, a rear surface piece 4 arranged on a rear surface 103 of the incised thyroid cartilage 101, and an end surface piece 5 connecting the front surface piece 2 and the rear surface piece 3 and abutting on an incision end surface 104 of the incised thyroid cartilage 101. As shown in FIG. 1 and FIG. 2, the pair of clamping portions 2 has a U-shape when viewed from the front, with a front surface piece 3, a rear surface piece 4, and an end surface piece 5 being integrally molded, and the respective clamping portions 2 are symmetrical in right and left.

The pair of clamping portions 2 has a role of engaging with each of the mutually opposed incision ends 105 of the incised thyroid cartilage 101. More specifically, as shown in FIG. 2, the front surface piece 3 has a bent region, and as shown in FIG. 2, when in use, the pair of clamping portions 2 can be engaged with the respective incision ends 105 by bending the respective front surface piece 3 of the pair of clamping portions 2. As shown in FIG. 2, when in use, the pair of clamping portions 2 has a J-shape.

Hereinafter, on the surface T opposite to the abutting surface S of the end surface piece 5 that abuts on the incision end 105, a side where the end surface piece 5 is joined to the front surface piece 3 is defined as one end side P, and a side where the end surface piece 5 is joined to the rear surface piece 4 is defined as the other end side Q.

Hereinafter, on the surface T opposite to the abutting surface S of the end surface piece 5 that abuts on the incision end 105, a direction from one end side P toward the other end side Q is defined as a vertical direction X, and on the opposite surface T, a direction orthogonal to the vertical direction X is defined as a horizontal direction Y.

Hereinafter, on the surface T opposite to the abutting surface S of the end surface piece 5 that abuts on the incision end 105, when the entire vertical direction X is defined as 1, the side located at a distance of, for example, 0.3 to 0.45 (0.4 in this example) away from one end side (line segment) P in the vertical direction X is defined as a line segment P′, and the side located at a distance of 0.55 to 0.7 away from one end side (line segment) P in the vertical direction X (0.6 in this example) is defined as a line segment Q′, the entire region in the horizontal direction Y enclosed between the line segment P′ and the line segment Q′ is defined as a central region C.

Hereinafter, on the surface T opposite to the abutting surface S of the end surface piece 5 that abuts on the incision end 105, when the entire distance in the horizontal direction Y is defined as 1, on one end side (line segment) P, a point located at a distance 0.3 to 0.45 away from the vertex p on the front side on the paper surface (0.4 in this example) is defined as a point p′, and a point located at a distance of 0.55 to 0.7 away from the vertex p on the front side on the paper surface (0.6 in this example) is defined as a point q′. The line segment pq between the point p′ and the point q′ is defined as a region R which is a region crossing in the vertical direction X from the one end side P toward the line segment P′.

Bridging portion 6

The bridging portion 6 bridges the pair of clamping portions 2 in the central region C and region R on the surface T on the opposite side.

Lengths of the Clamping Portion 2 and the Bridging Portion 6 15 The length of each clamping portion 2 is not particularly limited as long as it is capable of engaging the incision end of the incised thyroid cartilage, and for example, the length of the front surface piece 3 may be approximately 8 to 12 mm, the length of the end surface piece 4 may be approximately 1 to 4 mm, and the length of the rear surface piece 5 may be approximately 1 to 5 mm. In addition, the length of the bridging portion 6 (length in the connecting direction) may also be appropriately determined according to the distance between the incision ends of the incised thyroid cartilage, and may be, for example, approximately 1 to 4 mm.

### Materials of the Clamping Portion 2 and the Bridging Portion 6

Titanium is usually used as the material of the clamping portion 2 and the bridging portion 6. Titanium here is not limited to titanium as a pure metal, but is a concept that includes titanium alloys. More specific titanium includes Ti-6Al-4V and the like.

Method of Using the Dysphonia Treatment tool 1

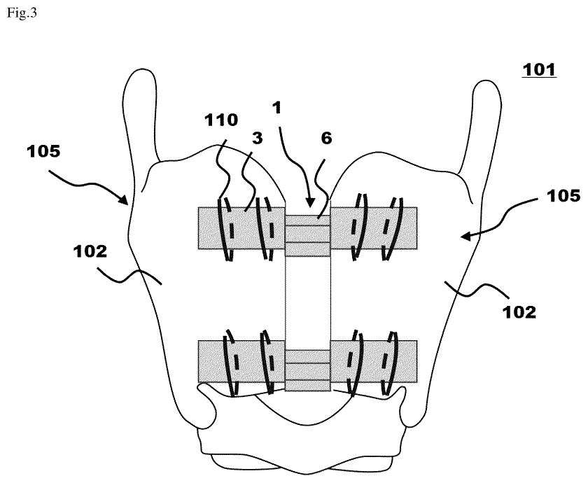

Next, a method of using the dysphonia treatment tool 1 will be described with reference to FIG. 3. FIG. 3 is an explanatory diagram that explains a method of using the dysphonia treatment tool 1 according to the present embodiment.

1. Engagement Method 35 First, the thyroid cartilage 101 is incised right in the middle, and the distance between the incision ends 105 that will improve phonation is determined. After determining the above-mentioned distance, the bent region of the front surface piece 3 is deformed so that the front surface piece 3 of the clamping portion 2 contacts the front surface 102 of the thyroid cartilage 101, and the clamping portion 2 is engaged with the incision end 40 105 so that the rear surface piece 4 contacts the rear surface 103 of the thyroid cartilage 101 and the end surface piece 5 contacts the incision end surface 104 of the thyroid cartilage 101, respectively.

### 2. Suturing Method

Here, in order to make the clamping portion 2 of the dysphonia treatment tool 1 more firmly engaged with the incision end 105, it is sutured and fixed using thread (for example, a nylon thread) 110. More specifically, as shown in FIG. 3, the front surface piece 3 of each clamping portion 2 and the front surface 102 side of the thyroid cartilage 101 are sutured and fixed with thread 110 so as to be pressed together.

### Comparison of the Dysphonia Treatment Tool 1 and Conventional Dysphonia Treatment Tool

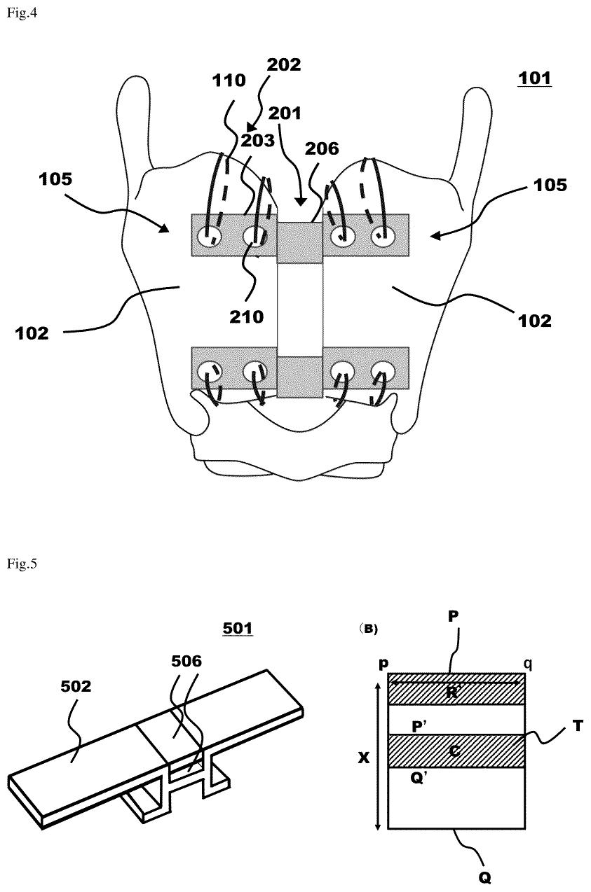

Next, the effects of the dysphonia treatment tool 1 will be explained in comparison with a conventional dysphonia treatment tool. First, the conventional dysphonia treatment tool will be explained with reference to FIG. 4. FIG. 4 shows a perspective view of a conventional dysphonia treatment tool 201 (for example, the dysphonia treatment tool disclosed in Patent Document 1).

Like the dysphonia treatment tool 1 according to the present embodiment, the dysphonia treatment tool 201 includes a pair of clamping portions 202 and a bridging portion 206. Each clamping portion 202 is the same as each clamping portion 2 of the dysphonia treatment tool 1, except that, as shown in FIG. 4, the front surface piece 203 is provided with a hole 210 for suturing the front surface piece 203 and the front surface 102 side of the thyroid cartilage 101 with thread (for example, nylon thread) 110. The bridging portion 206 is the same as the bridging portion 6 of the dysphonia treatment tool 1, except that it bridges the pair of clamping portions 202 only in the central region C shown in FIG. 1.

Next, a method of using the conventional dysphonia treatment tool 1 will be described with reference to FIG. 4. FIG. 4 shows an explanatory diagram that explains the conventional dysphonia treatment tool 201 and a method for using the same. First, the thyroid cartilage 101 is incised right in the middle, and the distance between the incision ends 105 that will improve phonation is determined. After the above distance has been determined, the engagement method is the same as that of the dysphonia treatment tool 1 of this embodiment. Next, as for the suturing method, as shown in FIG. 4, the front surface piece 203 and the front surface 102 side of the thyroid cartilage 101 are sutured and fixed with thread 110 through the hole 210 formed in the front surface piece 203 of each clamping portion 202.

Thus, the dysphonia treatment tool 201 shown in FIG. 4 can be firmly fixed to the thyroid cartilage and appears to be able to function to improve spasmodic dysphonia. However, when using the dysphonia treatment tool 201 shown in FIG. 4, there is a problem that the dysphonia treatment tool 201, particularly the clamping portion 202, is prone to breakage and does not have sufficient durability. Here, Patent Document 1 discloses that the hole 210 of the front surface piece 203 is designed in a specific way so as to disperse the elastic restoring force acting in the direction of closing the thyroid cartilage or the stress generated by vibrations during speaking and swallowing. However, the present inventors have found that even the dysphonia treatment tool 201 disclosed in Patent Document 1 does not yet sufficiently solve the problem that the clamping portion is prone to breakage, and there is room for improvement.

Therefore, after extensive research, the present inventors have found that the reason why the clamping portion 202 of the dysphonia treatment tool 201 as shown in FIG. 4 is prone to breakage is due to the bridge structure of the bridging portion 206. That is, the present inventors have found that, as a reinforcing structure to the bridging portion 206 of the dysphonia treatment tool 201 as shown in FIG. 4, by bridging the pair of clamping portions 2 in the region R on the surface T opposite to the end surface piece 5 as further shown in FIG. 1, the breakage of the clamping portion 202 can be effectively suppressed, resulting in excellent long-term durability.

It is to be noted that the dysphonia treatment tool of the present disclosure is not necessarily limited to the dysphonia treatment tool 1 shown in FIG. 1.

The pair of clamping portions are not particularly limited as long as they have a front surface piece arranged on the front surface of the incised thyroid cartilage, a rear surface piece arranged on the rear surface of the incised thyroid cartilage, and an end surface piece connecting the front surface piece and the rear surface piece and abutting on the incision end surface of the incised thyroid cartilage, and are engaged with each of the mutually opposed incision ends of the incised thyroid cartilage.

Although each of the pair of clamping portions 2 shown in FIG. 1 and FIG. 2 has a U-shape when viewed from the front, the shape of the clamping portions may be changed as appropriate as long as it is possible to engage each of the mutually opposed incision ends of the incised thyroid cartilage. However, from the standpoint of more reliably engaging with the incised thyroid cartilage, it is preferable that the shape of each of the pair of clamping portions 2 is U-shaped when viewed from the front.

In addition, the shapes of the front surface piece 3, the rear surface piece 4 and the end surface piece 5 of the clamping portion 2 shown in FIG. 1 and FIG. 2 are each strip-shaped plate-like, but the above shapes are not limited to this and may be, for example, the shapes disclosed in Patent Document 1.

In addition, in the clamping portion 2 shown in FIG. 1 and FIG. 2, the front surface piece 3, the rear surface piece 4 and the end surface piece 5 are integrally molded, but each may be composed of a separate member. However, since the front surface piece 3, the rear surface piece 4 and the end surface piece 5 are integrally molded, this is advantageous in that the clamping portion 2 is less likely to break.

In addition, although the pair of clamping portions 2 shown in FIG. 1 and FIG. 2 are symmetrical, they do not necessarily have to be symmetrical, and they may be asymmetrical as long as they are capable of engaging each of the mutually opposed incision ends of the incised thyroid cartilage described above.

In addition, the pair of clamping portions 2 shown in FIG. 1 and FIG. 2 are not provided with a hole in the front surface piece 5 for suturing to the front surface of the incision end of the thyroid cartilage, but the effects of the present disclosure can be achieved even if such hole is provided as in the conventional clamping portion 2. However, it is advantageous to not provide the above hole since this will result in greater durability.

The bridging portion 6 shown in FIG. 1 and FIG. 2 bridges the pair of clamping portions 2 in the regions of the central region C and the region R on the opposite surface T, but in the dysphonia treatment tool of the present disclosure, the bridging portion may have either of the following configurations (1) and (2). The effects of the present disclosure can be achieved in either of the following configurations (1) and (2).

(1) On the opposite surface to the abutting surface of the end surface piece, it is bridged in the region from the above one end side (on the opposite surface to the abutting surface of the end surface piece, the side where the end surface piece is joined to the front surface piece) to the above the other end side (on the opposite surface to the abutting surface of the end surface piece, the side where the end surface piece is joined to the front surface piece).

(2) On the opposite surface to the abutting surface of the end surface piece, it is bridged in the region from the above one end side (on the opposite surface to the abutting surface of the end surface piece, the side where the end surface piece is joined to the front surface piece) to the side in the midway from the one end side to the other end side (on the opposite surface to the abutting surface of the end surface piece, the side where the end surface piece is joined to the front surface piece).

However, the bridging portion of the dysphonia treatment tool according to the present disclosure is preferred in that, like the dysphonia treatment tool shown in FIG. 1 and FIG. 2, when the direction from one end side toward the other end side is defined as the vertical direction on the opposite surface to the abutting surface of the end surface, the bridging portion is bridged in the region from one end side to the central portion in the vertical direction on the opposite surface to the abutting surface of the end surface piece. This makes it possible to more firmly engage each of the mutually opposed incision ends of the incised thyroid cartilage, and to further suppress breakage of each clamping portion.

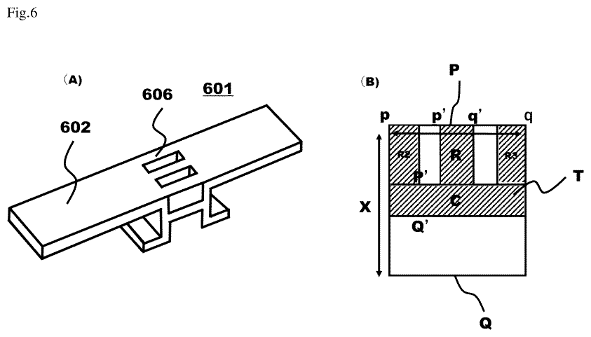

Modified examples of the bridging portion of the dysphonia treatment tool of the present disclosure are shown in FIG. 5 and FIG. 6.

FIG. 5 shows a perspective view (FIG. 5(A)) of another dysphonia treatment tool according to the present embodiment, and a front view of an abutting surface of an end surface piece that constitutes clamping portions of the dysphonia treatment tool and abuts on an incision end. As shown in FIG. 5(A) and FIG. 5(B), the dysphonia treatment tool 501 is the same as the dysphonia treatment tool 1 shown in FIG. 1 and FIG. 2, except that a bridging portion 506 bridges a pair of clamping portions 502 in the central region C and the region R′ (the surface opposed to the end surface in the longitudinal direction of the front surface piece 502) on the opposite side T.

FIG. 6 shows a perspective view (FIG. 6(A)) of the dysphonia treatment tool according to the present embodiment, and a front view of an incision end and end surface piece bent to the right, which constitutes the clamping portion of the dysphonia treatment tool. As shown in FIG. 6(A) and FIG. 6(B), the dysphonia treatment tool 601 is the same as the dysphonia treatment tool 1 shown in FIG. 1 and FIG. 2, except that the bridging portion 606 bridges the pair of clamping portions 602 in the central region C, as well as R, R2,R3 (region R2 and R3 are regions provided at a predetermined interval in the horizontal direction) on the opposite side T.

The effects of the present disclosure can also be achieved with the dysphonia treatment tool shown in FIG. 5 and FIG. 6.

The dysphonia treatment tool of the present disclosure is particularly suitable for use in treating a disorder known as spasmodic dysphonia.

### EXPLANATION OF REFERENCE SYMBOLS

dysphonia treatment tool . . . 1; clamping portion . . . 2; front surface piece . . . 3; rear surface piece . . . 4; end surface piece . . . 5; bridging portion . . . 6

1. A dysphonia treatment tool, comprising:
a pair of clamping portions that include a front surface piece arranged on a front surface of an incised thyroid cartilage, a rear surface piece arranged on a rear surface of the incised thyroid cartilage, and an end surface piece connecting the front surface piece and the rear surface piece and abutting on an incision end surface of the incised thyroid cartilage, and are engaged with each of mutually opposed incision ends of the incised thyroid cartilage; and
a bridging portion that bridges the pair of clamping portions,
wherein
on an opposite surface to the abutting surface of the end surface piece, when a side where the end surface piece is joined to the front surface piece is defined as one end side, and a side where the end surface piece is jointed to the rear surface piece is defined as other end side,
the bridging portion is bridging in a region from the one end side to the other end side, or in a region from the one end side to a side in a midway from the one end side to the other end side on the opposite surface to abutting surface of the end surface piece.

2. The dysphonia treatment tool according to claim 1, wherein
when a direction from the one end side toward the other end side is defined as a vertical direction on the opposite surface to the abutting surface of the end surface piece,
the bridging portion is bridging in a region from the one end side to a central portion in the vertical direction on the opposite surface to the abutting surface of the end surface piece.

3. The dysphonia treatment tool according to claim 2, wherein
when a direction orthogonal to the vertical direction is defined as a horizontal direction on the opposite surface to the abutting surface of the end surface piece,
the bridging portion
is bridging in a center of the horizontal direction from the one end side to a side from the one end side to the central portion on the opposite surface to the abutting surface of the end surface piece; and
is bridging in entire horizontal direction at the central portion.

4. The dysphonia treatment tool according to claim 1, wherein
the front surface piece, the end surface piece, the rear surface piece are integrally molded.

5. The dysphonia treatment tool according to claim 1, wherein
each of the clamping portions has a U-shape when viewed from the front.

6. The dysphonia treatment tool according to claim 2, wherein
the front surface piece, the end surface piece, the rear surface piece are integrally molded.

7. The dysphonia treatment tool according to claim 3, wherein
the front surface piece, the end surface piece, the rear surface piece are integrally molded.

8. The dysphonia treatment tool according to claim 2, wherein
each of the clamping portions has a U-shape when viewed from the front.

9. The dysphonia treatment tool according to claim 3, wherein
each of the clamping portions has a U-shape when viewed from the front.

10. The dysphonia treatment tool according to claim 4, wherein
each of the clamping portions has a U-shape when viewed from the front.

11. The dysphonia treatment tool according to claim 6, wherein
each of the clamping portions has a U-shape when viewed from the front.
